(19) United States
(12) Patent Application Publication
(10) Pub. No.: US 2015/0315142 A1
(43) Pub. Date: Nov. 5, 2015

(54) PROCESS TO OBTAIN IMIDAZOLINE MIXTURES FROM VEGETABLE OILS

(71) Applicants: CORROSION Y PROTECCION SA DE CV, CUERNAVACA MORELOS (MX); UNIVERSIDAD NACIONAL AUTONOMA DE MEXICO, Mexico City (MX)

(72) Inventors: Lorenzo Martínez GOMEZ, Cuernavaca Morelos (MX); Lorenzo Martínez Martínez DE LA ESCALERA, Mexico City (MX); Jorge Antonio Ascencio GUTIERREZ, Cuernavaca Morelos (MX); Jose Ignacio Regla CONTRERAS, Benito Juarez City (MX); Maria Patricia Shirley Demare NEGRETE, Mexico City (MX)

(21) Appl. No.: 14/321,103

(22) Filed: Jul. 1, 2014

(30) Foreign Application Priority Data
Apr. 30, 2014 (MX) MX/A/2014/005597

(51) Int. Cl. C07D 207/20; C07C 309/04

(52) U.S. Cl. CPC C07D 207/20; C07C 309/04

(57) ABSTRACT

A method to obtain imidazoline mixtures capable of providing considerable energy savings during the manufacture process due to a notable reduction of process temperature and lower requirements of chemical raw materials, and it allows for the direct use of vegetable oils, instead of fatty acids or their esters, producing imidazolines containing a (2-hydroxyethyl) group on position one of the ring and on position 2, a hydrocarbonated chain, from the fatty acids present in coconut oil, palm oil, jathrofa or castor. Likewise, the application of these mixtures as corrosion inhibitors has been established, in addition to quaternized imidazolines, which have applications as cationic surfactants, fabric softeners, hair conditioners and antistatic agents. It is an optimized method that compared to other known methods, uses substoichiometric proportions of aminoethylethanolamine and lower reaction temperatures, without using dehydrating agents or catalysts, or azeotropic distillation, which involves the use of highly flammable solvents.

### CROSS-REFERENCE TO RELATED APPLICATIONS

This application claims priority to Mexican Patent Application No. MX/A/2014/005597 filed Apr. 30, 2014, the disclosure of which is incorporated in its entirety by reference herein.

### TECHNICAL FIELD

The present invention patent finds its field of application within the chemical processes inherent to produce imidazoline mixtures from vegetable oils, such as coconut oil, palm oil, castor oil or jatropha for their use as corrosion inhibitors in the hydrocarbon transport, storage and processing industries. In addition, imidazolines can be transformed into quaternary salts through the reaction with a methylating agent, with applications as cationic surfactants, fabric softeners, hair conditioners, antistatic agents, and combinations of these products.

### BACKGROUND

The oil industry involves a wide variety of processes such as exploration, production, refining, petrochemistry, etc. Transport and distribution of liquid or gas petrochemical products is a process that requires an enormous network of steel pipelines with millions of kilometers of coverage around the world. Steel pipelines used for distribution and transport are affected by corrosion, due to the presence of water, carbon dioxide, anaerobic bacteria, hydrogen sulfide and other sulfur compounds that go along with hydrocarbons inside the pipelines. Corrosion of oil and gas pipelines may cause hydrocarbon leakage, which is responsible for severe economic and environmental damage in addition to endangering workers, people and properties around these pipelines.

Internal corrosion of pipelines and tanks can be drastically reduced by adding corrosion inhibitors, specifically imidazolines, which incorporate a hydroxyethyl chain on position one of the imidazolinic ring, and fatty acid chains on position two. Expansion of the worldwide pipeline network and increase of transport volumes are generating a demand on the manufacture of corrosion inhibitors, and consequently, motivating invention to create new forms of production. Considering that imidazoline manufacture processes haven't significantly evolved in the last decades, the modernization of imidazoline production is a relevant industrial issue.

An important strategy to inhibit interior corrosion of oil pipelines is the use of corrosion inhibitors, which are added directly to the stream of oil and gas. Imidazolinic inhibitors are highly efficient inhibiting corrosion in an acid environment; therefore, they are widely used to minimize H₂S and CO₂ induced corrosion (Hong, Sung, 2002).

This invention presents a new imidazoline manufacturing process, which is more cost-effective in the stages of its process, and makes better use of both energy and chemical additives.

Imidazolines and their quaternary salts have applications as corrosion inhibitors, surfactants, fabric softeners, air-conditioners or antistatic agents. This type of imidazolines was described since the 40s (U.S. Pat. No. 2,200,815). In that regard, Divya BAJPAI and V. K. TYAGI, in their review article: “Fatty Imidazolines: Chemistry, Synthesis, Properties and Their Industrial Applications”, Journal of Oleo Science; Vol. 55, No. 7, 319-329 (2006), describe experimental evidence of imidazoline quaternary salts being used for the applications mentioned above.

Coconut oil is a natural mixture of triglycerides, constituted by fatty acids with 8 to 18 carbon atoms, with the following percentage composition of fatty acids: lauric acid, 48-52%; myristic acid, 18-22%; palmitic acid, 8-12%; capric acid, 4-8%; caprilyc acid, 3-7%; oleic acid, 4-8%; stearic acid, 2-3% and linoleic acid, 1-2%. Palm oil has the following approximate percentage composition of fatty acids:

Several methodology alternatives for the synthesis of this type of imidazolines have been described, that generally involve the reaction of free fatty acids of 8 to 20 carbon atoms (U.S. Pat. No. 5,214,155, 1993) or biodiesel (U.S. Pat. No. 4,709,045, 1987; Corrosion Science 59 (2012) 42-54) with poliamines, such as aminoethylethanolamine or diethylenetriamine at temperatures around 200° C. For example, U.S. Pat. No. 4,182,894 (1980) claims the preparation of this type of compounds from free fatty acids with molar excesses of aminoethylethanolamine, at temperatures of 180 to 250° C. Other patents that describe this process with several variations, are the following: CN 102093296 (2012), KR 2012117201 (2012), CN 102093296 (2011), JP 54063077 (1979). The use of microwaves has also been described (Synlett 2003, 12, 1847-1849).

Patent EP 0604726 describes a process for the quaternization of triethanolamine and imidazoline amide fatty acid esters, where the resulting mixtures are used as active components for cloth detergents and softeners. This patent does not mention the use of dimethyl sulfate as a methylating agent to obtain quaternary imidazoline salts.

Patent EP 1105449 refers to amphoteric derivatives of aliphatic polyamines, such as diethylenetriamine or triethylenetetramine, where polyamines are first treated with fatty acids, esters or triglycerides from animal, vegetable or synthetic sources to form diamides or imidazolines. These compounds are then reacted with unsaturated or halogenated carboxylic acids, carboxylated epoxy compounds or acid anhydrides (such as acrylic acid, itaconic acid, chloroacetic acid, maleic anhydride, octadecenyl anhydride) to form the various amphoteric structures. This invention does not consider the reaction of the vegetable oils mentioned above to formulate imidazolines using aminoethylethanolamine (AEEA) in substoichiometric proportions.

Patent JP2007-277374 describes a method to produce biodiesel using the transesterification and esterification reactions of vegetable or animal oils and fatty acids with an alcohol, induced by microwave. However, it does not describe the procedures related to the production of imidazolines from biodiesel by aminolysis reaction with AEEA.

Patent JP2010-013511 introduces a method for the production of biofuels, from the fatty acids obtained from the plant known as Xanthoceras sorbifolia Bunge. The method to produce the biodiesel includes the extraction of the oil from the seed of Xanthoceras sorbifolia Bunge, a transesterification step by adding methanol to the vegetable oil in the presence of an acid or a basic catalyst, as well as an additional step involving methyl ester separation. Similarly, it does not consider the use of such oils to produce corrosion inhibitors nor procedures conducive to such purposes.

Patents PA/a/2002/006434 and KR20120117201, US20040144957, US20040200996, describe the production of corrosion inhibitor imidazolines derived from vegetable oil fatty acids. In the procedures described, these patents do not consider the use of substoichiometric amounts of aminoethylethanolamine for the production of residual amine-free imidazolines.

The invention with registration number GB989743 is related to the production of imidazolines from fatty acid mixtures and fatty acid esters to produce sulfobetaines, whose general formula is based on a basic tertiary nitrogen atom, with a mixture of propanesultone with 1,2 disubstituted imidazoline. The purpose of this invention is to introduce a fast and direct procedure for the production of these compounds for applications such as detergents and fabric softeners. However, it does not consider a procedure for the aforementioned imidazoline to be used as a corrosion inhibitor.

Patent GB1187131 introduces a method for the production of imidazolines and their byproducts, using a reaction procedure consisting of heating a fatty acid such as lauric acid or coconut fatty acid of a (RCOOH) structure, where R is a hydrocarbon chain of at least eight carbon atoms. This fatty acid reacts with a diamine NH₂(CH₂)₂NHR₁, where R₁ is an alkyl chain of 2 to 4 carbon atoms. These imidazolines do not contain in position one of the imidazoline ring a 2-hydroxyethyl chain, nor do they contain, on position two, a hydrocarbonated chain.

Invention GB985321 describes a process for the treatment of fabrics by using amphoteric softening agents derived from sulphated imidazolines having on position two, an alkyl group with a length of 12 to 18 carbon atoms. Likewise, this patent does not describe any process for the production of imidazolines from the triglycerides present in vegetable oils such as coconut, castor, jathrofa or palm oil.

Patents with registration numbers GB815784 and GB814246 claim extracting and using quaternary ammonium salts derived from non-aromatic carboxylic acids in preparations of hair shampoo, which have foaming properties, and are characterized by containing at least one non-aromatic hydrocarbon residue of more than seven carbon atoms linked to one quaternary nitrogen atom. These patents do not consider the inclusion of methyl imidazolines for the production of the aforementioned quaternary salts, nor the use of a methylating agent for their production, such as dimethyl sulfate.

Other inventions found in the literature regarding production of imidazoline, are found in patents with registration numbers U.S. Pat. No. 2,200,815, U.S. Pat. No. 2,161,938, U.S. Pat. No. 4,161,604, U.S. Pat. No. 4,182,894, U.S. Pat. No. 4,709,045, U.S. Pat. No. 5,013,846, U.S. Pat. No. 5,049,315, U.S. Pat. No. 5,144,040 and U.S. Pat. No. 5,214,155. None of these patents mentions the direct reaction of vegetable oils such as the ones present in coconut, castor, jathrofa or palm, with substoichiometric proportions of aminoethylethanolamine for the production of amine-free imidazolines that have, on position 1 of the imidazoline ring, a 2-hydroxyethyl chain, and on position 2, a hydrocarbonated chain of 7 to 19 carbon atoms, nor do they consider the application of these imidazolines for their use as corrosion inhibitors in the hydrocarbon transport and storage industries.

### SUMMARY

The imidazolines obtained through the process of this invention, contain, on position one of the imidazoline ring, a 2-hydroxyethyl chain, and a hydrocarbonated chain on position two an R group, of 7 to 19 carbon atoms, either saturated, unsaturated or hydroxylated. Likewise, imidazolines can form the corresponding quaternary salts, through a reaction with a methylating agent, such as for example dimethyl sulfate.

The process to obtain imidazoline mixtures through the direct reaction of vegetable oils, mainly coconut, palm, jatropha or castor has considerable advantages compared to the processes that are prevalent in industrial practice. The process provides considerable energy savings during manufacture due to a notable temperature reduction during the process. Likewise, the process provides lower raw material requirements and allows for the direct use of vegetable oils, instead of fatty acids or their esters.

Our process consists of direct aminolysis at 140-150° C. of the triglycerides present in vegetable oils, using 85-90% of the stoichiometric amount of aminoethylethanolamine required. Once the aminolysis process is completed, an incremental vacuum between 20 and 225 Torr is applied, until the stoichiometric amount of water is removed, with the consecutive construction of the corresponding imidazolines. As an option, in order to obtain the quaternary salts of the imidazolines, at the end of the process, a heat treatment with an alkylating agent such as dimethyl sulfate between 50° C. and 60° C. may be performed.

The cationic or neutral imidazoline mixtures obtained have applications as corrosion inhibitors in the hydrocarbon storage and transport industry, and the quaternary imidazolines have applications as cationic surfactants, fabric softeners, hair conditioners or antistatic agents.

### BRIEF DESCRIPTION OF THE DRAWINGS

FIG. 1. Corrosion inhibition test of an imidazoline mixture obtained from castor oil at a concentration of 5, 10 and 25 ppm.

FIG. 2. Corrosion inhibition test of an imidazoline mixture obtained from palm oil at a concentration of 5, 10 and 25 ppm.

FIG. 3. Corrosion inhibition test of an imidazoline mixture obtained from coconut oil at a concentration of 10 ppm.

### DETAILED DESCRIPTION

The present invention document describes a method through which it is possible to obtain imidazoline mixtures, characterized by the structure of their general formula:

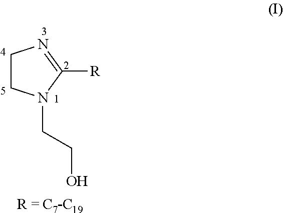

Where position one of the imidazoline ring, shows a 2-hydroxyethyl chain and a hydrocarbonated chain of fatty acids on position 2 (R), of 7 to 19 carbon atoms, either saturated, unsaturated or containing a hydroxyl group, present on at least one of the oils contained in the group of coconut, palm, jatropha and castor.

The process consists on heating a mixture of vegetable oil and 85-90% of the molar equivalent of aminoethylethanolamine, at temperatures ranging between 140-150° C. and atmospheric pressure, for 1 to 3 hours or until the chromatographic analysis shows the complete consumption of the vegetable oil, and then for 4-6 hours at a pressure ranging between 20 and 225 torr, until obtaining the stoichiometric amount of water, with the consecutive cycling of the corresponding imidazolines. Going into further detail, the process for the production of imidazolines, that can be cationic or neutral, can be described in the following steps:

Step 1: heating the vegetable oil and the aminoethylethanolamine at a temperature that ranges between 140° C. to 150° C. at atmospheric pressure, for a time that may be 1 to 3 hours, until consuming the triglycerides present in the vegetable oil, following the course of the reaction by thin layer chromatography, using heptane-ethyl acetate 9:1 as the mobile phase.

Step 2: gradually reducing the pressure between 20 and 225 Torr for 4 to 8 hours, until transforming the intermediate hydroxyethyl aminoethyl amides into the corresponding imidazoline mixtures, following the course of the reaction by thin layer chromatography using dichloromethane-methanol 7:3 as the mobile phase, adding 50 microliters of concentrated ammonium hydroxide for every milliliter of mobile phase.

In order to obtain a mixture of quaternary imidazolines, dimethyl sulfate (the same molar proportion of the amine used) is slowly added at a temperature ranging between 50-60° C., and heated afterwards at a temperature ranging between 50 and 60° C. for one hour, following the course of the reaction (by) thin layer chromatography using dichloromethane-methanol 7:3, as mobile phase, adding 50 microliters of concentrated ammonium hydroxide for every milliliter of mobile phase.

A mixture of quaternary imidazolines is therefore obtained, characterized by the structure of the general formula:

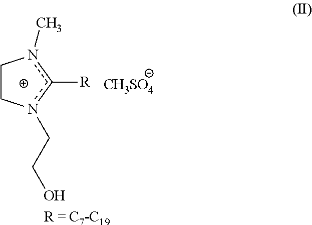

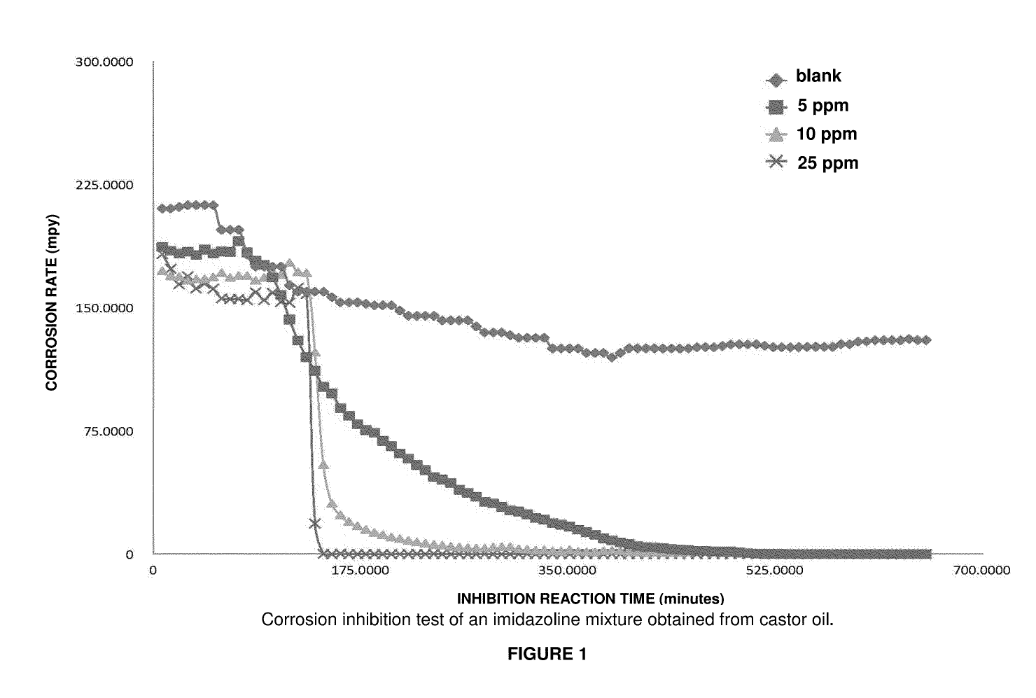

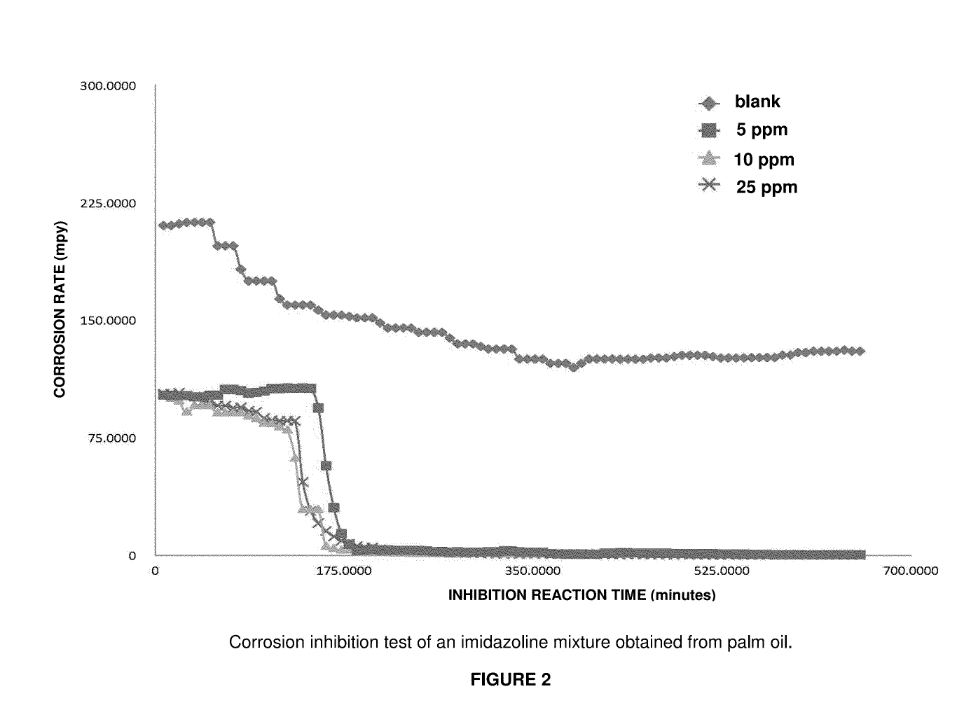

The imidazoline mixtures produced through the process described in this invention, and specifically the ones obtained from castor oil, showed important corrosion inhibition effects on steel in contact with samples of water at different concentrations of chlorides, carbon dioxide and H₂S. Likewise, corrosion inhibition presented itself when injecting doses of imidazolines in brackish water and crude oil. The imidazoline concentrations that showed corrosion inhibition were 5, 10, 15 ppm and higher concentrations. The charts on FIG. 1 and FIG. 2 illustrate corrosion inhibition effects of the imidazolines obtained from castor oil and palm oil at a concentration of 5, 10 and 25 ppm.

For practical purposes of the stoichiometric calculation, a molecular weight of 639 g is considered for coconut oil; 933 for castor oil, and for palm oil a molecular weight of 891 g.

A general scheme of the chemical reactions that take place during the process of the present invention is shown below, from vegetable oils down to the corresponding quaternization that occurs through the reaction with dimethyl sulfate.

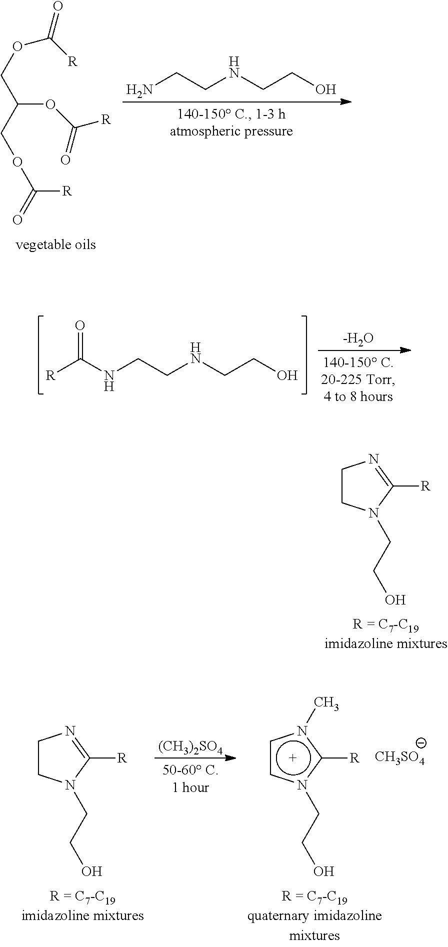

The advantages of this invention compared to what has been described in the patent literature are: A) the triglycerides present in coconut oil, castor or palm are used, instead of using fatty acids or their corresponding esters; B) substoichiometric proportions of aminoethylethanolamine are used, producing amine-free imidazolines with better environmental performance, reducing toxicity in marine environments; C) dehydrating agents or solvents are not required for azeotropic distillation; D) the use of lower reaction temperatures, (between 140 and 150° C.), lower than the ones reported in the literature, produces a crude mixture of imidazolines that provides excellent corrosion inhibition capabilities.

### Best Known Method to Develop the Invention

These examples are potentially including but not limited to, given that any technician will understand that there are variables that fall within the scope of this invention.

The following description details some preferred inclusions, among others both existing and obvious, in order to illustrate the approach described, and specifically on the subject disclosed herein, without any implication of limiting similar processes:

### Process Example 1

### 1-Hydroxyethyl 2-Alkyl-Coconut Oil Imidazolines

A mixture of 100 g coconut oil and 52.3 g (497.1 mmol) of aminoethylethanolamine was heated at 140-142° C. in a controlled temperature oil bath for three hours, verifying total oil consumption by TLC, in a hexane: MTBE 9:1 system. The system pressure was gradually reduced down from atmospheric pressure to 20-225 Torr, maintaining the same temperature and pressure for eight additional hours, until the complete disappearance of intermediate amides (TLC: CH₂Cl₂:MeOH:NH₄OH 80:20:1;). The mixture was allowed to cool down to 60° C. and transferred to a storage container with an airtight seal. 139 g of the imidazoline mixture was obtained.

### Process Example 2

### 1-Hydroxyethyl 2-Alkyl-3-Methyl Methyl Sulfate Coconut Oil Imidazolines

The imidazoline mixture was prepared as described above, and 49 mL (65.5 g, 519.6 mmoles) of dimethyl sulfate was slowly added at 50-60° C. The mixture was heated to 70° C. for three hours, verifying the completion of the reaction by TLC (CH₂Cl₂:MeOH:NH₄OH 80:20:1) and was concentrated at reduced pressure, in order to obtain 202.5 g of the coconut oil methylimidazoline methylsulfates.

### Process Example 3

### 1-Hydroxyethyl-2-Alkyl-Castor Oil Imidazolines

208.83 g (2,005 Mol) of aminoethylethanolamine was loaded into a three-necked flask equipped with mechanical stirring and a thermometer. 672.0 g (0.7542 Mol) of castor oil was heated and stirred for six hours, observing the full consumption of the oil by thin layer chromatography, using 7:3 ethyl acetate-heptane as the mobile phase and visualizing with iodine vapors. The mixture was allowed to cool down at room temperature and heating was started again at a vacuum of 38-40 Torr at 140-145° C. for two hours verifying the formation of Imidazolines by thin layer chromatography, using 7:3:0.5 Dichloromethane-Isopropanol-ammonium hydroxide as a mobile phase, visualizing initially with ultraviolet light followed by iodine vapors. 860 g of a thick oil was obtained.

### Distinctions for Dissemination

The advantages of this invention, among others, which are apparent for individuals with medium knowledge about the previous procedure and technique, are mentioned below:

Better energy efficiency: by operating at lower temperatures (between 140-150° C.) compared to most processes, which report temperature ranges of up to 300° C.

Fewer steps: does not require isolation of free fatty acids present in vegetable oils nor the generation of their methyl esters.

Reduced chemical compound consumption: due to the reduction of the number of chemical reactions, and the use of substoichiometric amounts of aminoethylethanolamine.

The above represents an additional advantage, given that the mixture of imidazolines obtained remains exempt of free aminoethylethanolamine which is toxic for marine fauna.

### Corrosion Inhibition Test

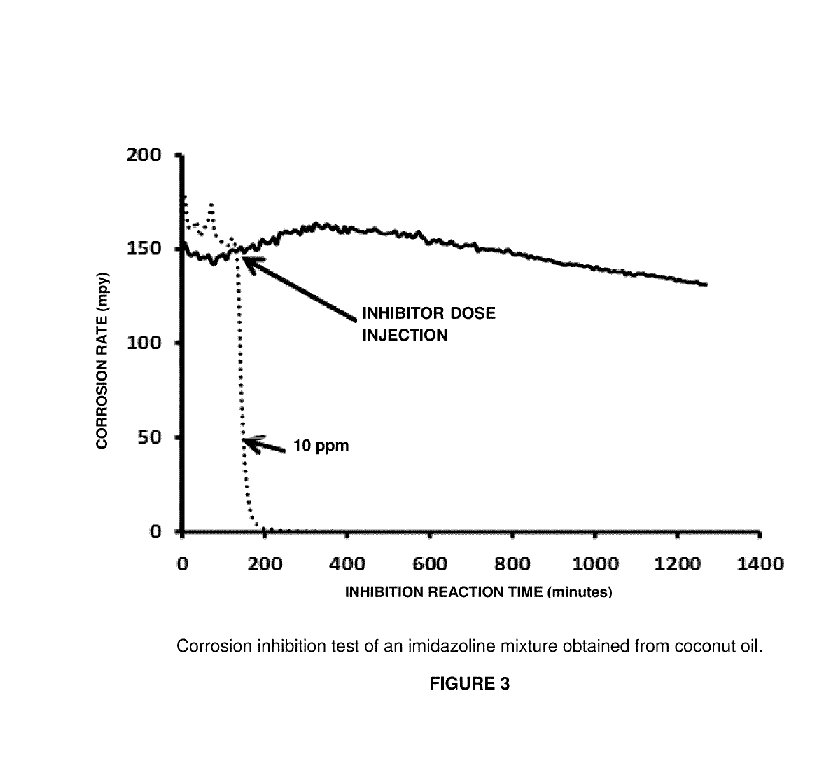

FIG. 3 is a chart that shows an example of the corrosion mitigation effect obtained with a mixture of coconut oil imidazolines using the linear polarization resistance method according to standard ASTM G3-89(2010), measured in real-time. The vertical axis records corrosion speed in mils of an inch per year, and the horizontal axis records time, in minutes, to establish the transition of the corrosion phenomenon of steel in contact with oil-brackish water mixtures, a typical flow condition in oil pipelines. The solid curve represents the corrosion phenomenon in absence of the imidazoline mixture, averaging an annual metal loss of close to 4 mm per year equivalent to 150 MPA (mils of an inch/year), which is a level of corrosivity that can have catastrophic consequences. The dotted curve represents the sudden reduction of corrosion speed that is accomplished a few minutes after the addition of the imidazoline mixture developed under this process at a concentration of 10 ppm. Once having added a dose of between 5 and 25 ppm of the inhibitor manufactured following the process reported herein, corrosion was reduced from 4 mm to less than 2.5 microns per year.

Despite the fact that the above description was made taking into account the preferred modalities of the invention, it must be taken into account by experts in the field, that field that any modification of form or detail must fall within the boundaries of the spirit and scope of the present invention. The terms under which this document was written must be taken under wide and non-restrictive terms. The materials, form and description of the elements, may be susceptible to variations as long as this does not entail variations of the essential features of the model.

What is claimed is:

1. A method to obtain mixtures of vegetable oil imidazolines, with the following general formula structure: 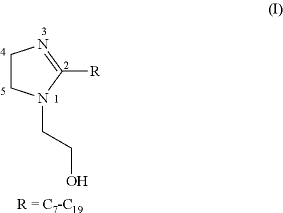
Where position 1 of the imidazoline ring shows a hydroxyethyl chain and position 2 shows one of the fatty acid chains present in at least one of the oils of the group of coconut, palm, jatropha and castor; where said method entails the following steps:
Step 1: heating the vegetable oil and the aminoethylethanolamine at a temperature ranging from 140° C. to 150° C. at atmospheric pressure, for a time that may go from 1 to 3 hours, until consuming the entirety of the triglycerides present in the vegetable oil, following the course of the reaction by thin layer chromatography, using ethyl heptane-acetate 9:1 as the mobile phase.
Step 2: gradually reduced down from atmospheric pressure to 20-225 Torr, for a time that may go from 4 to 8 hours, until transforming the intermediary hydroxyethylaminoethylamides into the corresponding imidazoline mixtures following the course of the reaction by thin layer chromatography, using dichloromethane-methanol 7:3 as the mobile phase, adding 50 microliters of concentrated ammonium hydroxide for every milliliter of mobile phase.

2. The method, as established in claim 1, distinguishes itself because it uses substoichiometric amounts, between 85 and 90% molar equivalent of aminoethylethanolamine, producing residual amine-free imidazolines.

3. According to claim 1, the method includes an optional step to obtain quaternary salts from the imidazolines obtained by this method, with the following general structure: 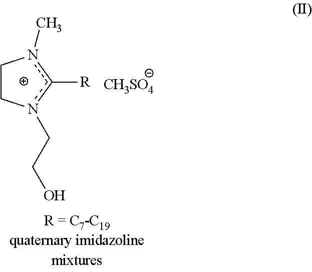
Where dimethyl sulfate (the same molar proportion of the amine used), is slowly added to the imidazoline mixture at a temperature ranging between 50-60° C.; heating between 50 and 60° C. for one hour, following the course of the reaction by thin layer chromatography using dichloromethane methanol 7:3 as mobile phase; adding 50 microliters of concentrated ammonium hydroxide for every milliliter of mobile phase.

4. An imidazoline mixture from vegetable oils obtained according to the method from claim 1, with the following general formula structure: 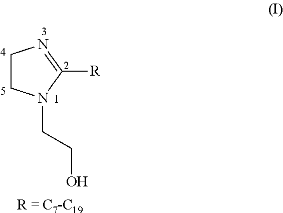
Where a hydroxyethyl chain can be found on position one of the ring, and position 2 shows one of the fatty acid chains present in at least one of the oils of the group of coconut, palm, jatropha and castor.

5. The mixture obtained according to claim 4, distinguishes itself because it is used as a corrosion inhibitor for metallic structures.

6. According to claim 5, the mixture is extremely useful because metallic structure corrosion is provoked by at least one of the compounds in the group consisting of hydrocarbons, crude oils, condensates, natural gas, refined oil products with second water phases of various concentrations of chlorides, carbon dioxide, hydrogen sulfide and combinations of corrosive chemicals derived from these combinations.

7. The quaternary salts of the imidazolines obtained according to claim 3, have the following general formula structure: 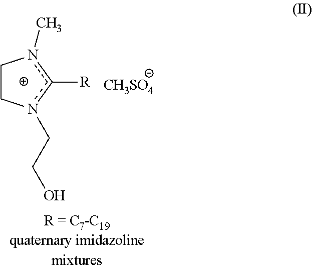
Where dimethyl sulfate is slowly added to the imidazoline mixture obtained at a temperature of 50-60° C. (the same molar proportion of the amine used), and heating between 50 and 60° C. for one hour, following the course of the reaction by thin layer chromatography using dichloromethane methanol 7:3 as mobile phase; adding 50 microliters of concentrated ammonium hydroxide for every milliliter of mobile phase.

8. The quaternary salts according to claim 7 are suitable to be used as cationic surfactants, fabric softeners, hair conditioners, antistatic agents, and other combinations of these products.
